(19) United States
(12) Patent Application Publication
(10) Pub. No.: US 2012/0189564 A1
(43) Pub. Date: Jul. 26, 2012

(54) COMPOSITIONS OF VITAMIN C DERIVATIVE AND PORTULACA EXTRACT

(75) Inventors: Yelena Zolotarsky, Springfield, NJ (US); Gregory Torchiana, Phoenixville, PA (US); Yun Ha Hwang, Chungcheongnam-do (KR)

(73) Assignee: PRESPERSE CORPORATION, Somerset, NJ (US)

(21) Appl. No.: 13/353,458

(22) Filed: Jan. 19, 2012

Related U.S. Application Data
(60) Provisional application No. 61/434,654, filed on Jan. 20, 2011.

(51) Int. Cl. A61K 8/97; A61Q 19/02

(52) U.S. Cl. 424/62

(57) ABSTRACT

Compositions including Portulaca extract and a vitamin C derivative selected from ascorbyl glucoside (ASG), magnesium ascorbyl phosphate an ethyl ascorbic acid. The compositions can be employed as an inhibitor to melanin formation and used as an additive to a cosmetic or pharmaceutical formulation.

### CROSS-REFERENCE TO RELATED APPLICATIONS

This application claims the benefit of U.S. Provisional Patent Application Ser. No. 61/434,654 filed Jan. 20, 2011, the entirety of which is incorporated herein by reference.

### FIELD OF THE INVENTION

The present invention relates in general to compositions having enhanced skin-whitening properties, and particularly to compositions including vitamin C derivatives and Portulaca extract.

### BACKGROUND OF THE INVENTION

Oxygen radicals, which are formed in the skin during its exposure to sunlight, are believed to cause skin damage and reddening of the skin. Portulaca Oleracea (“Portulaca”), is an annual herbaceous plant which originates from the region extending from the western Himalayas to southern Russia and Greece. It is now found all over the world. Portulaca is eaten as a salad and vegetable around the world and used medicinally for a variety of conditions that include headache, stomach ache, painful urination, enteritis, mastitis, lack of milk flow in nursing mothers and in postpartum bleeding. Externally it is used to treat burns, earache, insect stings, inflammations, skin sores, ulcers, pruritis, eczema and abscesses. Leung & Foster, 1996.

Portulaca contains numerous common nutrients including vitamins; minerals; fatty acids, glutathione, glutamic acid and aspartic acid. Leung & Foster, 1996. Recent research has shown that Portulaca is a rich source of omega-3 fatty acids, which are thought to be important in preventing heart attacks and strengthening the immune system. Brown, 1995 The whole plant contains carotene, vitamins C, B₁, B₂ PP, calcium, magnesium, sodium, and potassium salts, nicotinic and oxalic acids; noradrenaline, and the biflavonoid liquiritin. World Health Organization, 1990.

Portulaca Oleracea extract (“Portulaca extract”) is an extract obtained from the whole plant. It is believed to have anti-inflammation and anti-irritation effects. The extract is used as a basic ingredient for cosmetics, toiletries and pharmaceuticals for use in the treatment of dandruff, atopic dermatitis, erysipelas, hair loss, acne and insect bites.

### SUMMARY OF THE INVENTION

Compositions in accordance with the present disclosure include Portulaca extract and a vitamin C derivative selected from ascorbyl glucoside (ASG), magnesium ascorbyl phosphate and ethyl ascorbic acid.

It is an objective of this invention to provide compositions that can be employed as an inhibitor to melanin formation.

It has surprisingly been found that compositions including a blend of ascorbyl glucoside (ASG) and Portulaca extract provide improved melanin formation inhibition. This finding is especially surprising in view of the fact that Portulaca extract by itself shows almost no melanin formation inhibition. The synergistic effect of the combination of ASG with Portulaca extracts on melanin formation inhibition is unexpected.

Test results indicate that ASG solution (ASG in water) shows melanin formation inhibition in a dose dependent manner. Portulaca extract (solution in water) shows almost no melanin formation inhibition. However, surprisingly, compositions containing a blend of ASG powder and Portulaca extract demonstrate the same activity as ASG solution alone, despite containing a lower percentage of ASG.

In one embodiment a composition including ASG and Portulaca extract is provided in the form of an additive which can be used in cosmetic, pharmaceutical or other formulations.

It is a further objective that the compositions include ASG and Portulaca extract, and optionally at least one antioxidant and one preservative.

In one embodiment a composition including magnesium ascorbyl phosphate and Portulaca extract is provided in the form of an additive which can be used in cosmetic, pharmaceutical or other formulations.

It is a further objective that the compositions include magnesium ascorbyl phosphate and Portulaca extract, and optionally at least one antioxidant and one preservative.

In one embodiment a composition including ethyl ascorbic acid and Portulaca extract is provided in the form of an additive which can be used in cosmetic, pharmaceutical or other formulations.

It is a further objective that the compositions include ethyl ascorbic acid and Portulaca extract, and optionally at least one antioxidant and one preservative.

Compositions of the present invention are useful in formulations including skin care products, color cosmetics, and pharmaceutical products, especially skin whitening products used for fading and/or lightening age spots, liver spots, freckles and uneven skin tone.

### BRIEF DESCRIPTION OF THE DRAWINGS

FIG. 1 is a graphical representation of the degree of melanin formation inhibition of samples in accordance with the present disclosure; and

FIG. 2 is a graphical representation of the boosting effect of Portulaca Extract with ASG in accordance with the present disclosure.

### DETAILED DESCRIPTION OF THE INVENTION

In the following description, for purposes of explanation, specific numbers, materials and configurations are set forth in order to provide a thorough understanding of the invention. It will be apparent, however, to one having ordinary skill in the art that the invention may be practiced without these specific details. In some instances, well-known features may be omitted or simplified so as not to obscure the present invention. Furthermore, reference in the specification to “one embodiment” or “an embodiment” means that a particular feature, structure or characteristic described in connection with the embodiment is included in at least one embodiment of the invention. The appearances of the phrase “in one embodiment” in various places in the specification are not necessarily all referring to the same embodiment.

Compositions are provided including vitamin C derivatives and Portulaca extract. Vitamin C derivatives may be selected from ASG, magnesium ascorbyl phosphate and ethyl ascorbic acid.

Ascorbyl glucoside (2-0-\acute{α}-D-glucopyranosyl-L-ascorbic acid, C₁₂H₁₈O₁₁), also known as ascorbic acid 2-glucoside or ASG, is a vitamin C derivative obtained by the condensation of ascorbic acid with glucose and has the chemical structure:

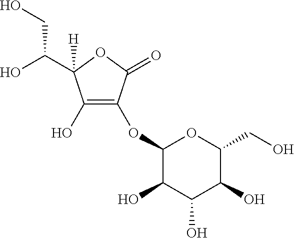

It is believed the C2 hydroxyl group of ascorbic acid is the primary site of natural vitamin C's beneficial behavior, however, this is the site where vitamin C is degraded. The glucose protects vitamin C from high temperatures, pH, metal ions and other mechanisms of degradation. Ascorbyl glucoside has been used as an additive in skin care creams and lotions. It was originally developed as a quasi-drug cosmetic product in Japan to lighten the overall tone of skin and reduce the pigmentation in age spots and freckles. It is now also used as an additive in products for brightening dull-looking skin, reversing the effects of aging and in sunscreen products for protection. When products containing ascorbyl glucoside are used on the skin, the action of \acute{α}-glucosidase gradually releases vitamin C, providing the benefits of vitamin C over a prolonged period of time. Ascorbyl glucoside is more soluble than natural vitamin C and is stable over a wide range of pH values, especially at pH 5.0-7.0 which is typically used for formulation of skin products. Ascorbyl glucoside slowly releases vitamin C, which acts as a free radical scavenger, thereby reducing the amount of skin inflammation and roughness.

ASG is available commercially for example from Presperse Corp. of Somerset, N.J. as AS-G powder and from Kabushiki Kaisha Hayashibara Corporation of Japan as AA2G® ascorbyl glucoside powder (CAS number 129499-78-1). The purity of the ASG for use in the composition is preferably greater than 98%.

Magnesium ascorbyl phosphate (MAP) has the general chemical formula C₆H₇O₉P:Mg and the following structure:

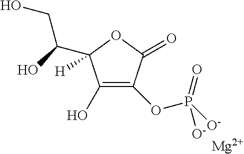

It is a water-soluble salt of vitamin C which when applied to the skin is converted to vitamin C by the action of enzymes in the skin. Magnesium ascorbyl phosphate is available commercially for example from Presperse Corp. as Ascorbyl PM (CAS#113170-55-1). The purity of the magnesium ascorbyl phosphate for use in the compositions disclosed herein is preferably at least 95%. In some embodiments a mixture of magnesium ascorbyl phosphate and about 1 to about 3% by weight of sodium citrate may employed in the disclosed compositions. Sodium citrate may be combined with magnesium ascorbyl phosphate to increase the solubility of the magnesium ascorbyl phosphate.

Ethyl ascorbic acid is a stable vitamin C derivative with melanin inhibition properties. Ethyl ascorbic acid is available commercially for example from Corum Inc. of Taipei, Taiwan as Corum 9515.

Portulaca extract is a colorless to light-yellow liquid which can be used as an additive in cosmetic, pharmaceutical or other formulations. Portulaca extract may contain Portulaca Oleracea in concentrations of from about 0.2 to about 1.0% by weight, and may contain from about 50 to about 99% by weight of water. Portulaca extract may also include glycols selected from one or more of butylene glycol, propylene glycol, ethylene glycol, glycerin, etc. Glycols may be present in an amount of about 1.0 to about 50% by weight. Portulaca extract is commercially available for example from Bioland of South Korea and Presperse Corp. of Somerset, N.J. Portulaca extract available commercially from Presperse Corp. contains approximately 0.2 to 0.6% by weight Portulaca Oleracea, up to 70% by weight water and up to 30% by weight butylene glycol.

The compositions disclosed herein may further contain one or more additives such as antioxidants, preservatives, excipients and the like. Suitable antioxidants include but are not limited to Sea Buckthorn berry extract, green tea extract, ferulic acid, coenzyme Q10. Sea Buckthorn berry extract is commercially available from Aromtech Ltd of Tornio Finland as Shajifen Sea Buckthorn Phenolic extract (CAS 90106-68-6, 9050-36-6), which contains Sea buckthorn extract (50-70%) and maltodextrin DE 6 (30-50%).

Suitable preservatives include but are not limited to Euxyl PE 9010, phenoxyethanol, propylene glycol, butylene glycol, and potassium sorbate. Euxyl PE 9010 (phenoxyethanol and ethylhexylglycerin) is commercially available from Schulke & Mayr GmbH of Norderstedt, Germany.

The composition may further include a base such as KOH, NaOH, TEA etc. The composition may further contains magnesium ascorbyl phosphate, soy extract, rice extract, resveratrol, niacin, algae extract, etc., as well as other anti-inflammatory active ingredients such as bisabolol, apple extract, meadowfoam extract, honeysuckle extract, sea whip extract etc.

Compositions in accordance with the present disclosure may contain from about 2-98% by weight vitamin C derivative selected from ASG, magnesium ascorbyl phosphate and ethyl ascorbic acid and from about 2-98% by weight Portulaca extract. In one embodiment compositions may include from about 20%-60% vitamin C derivative and about 40%-80% Portulaca extract. In another embodiment a composition in accordance with the disclosed subject matter includes about 45% vitamin C derivative and about 45% Portulaca extract. In another embodiment a composition includes about 40% vitamin C derivative and about 50% Portulaca extract. In another embodiment a composition includes about 35% vitamin C derivative and about 55% Portulaca extract. In another embodiment a composition in accordance with the disclosed subject matter includes about 30% vitamin C derivative and about 60% Portulaca extract. In another embodiment a composition includes about 50% vitamin C derivative and about 40% Portulaca extract. In another embodiment a composition includes about 55% vitamin C derivative and about 35% Portulaca extract. In another embodiment a composition includes about 60% vitamin C derivative and about 40% Portulaca extract.

In a preferred embodiment a composition in accordance with the present disclosure may include from about 20%-60% ASG and about 40%-80% Portulaca extract. In another embodiment a composition in accordance with the disclosed subject matter includes about 45% ASG and about 45% Portulaca. In another embodiment a composition includes about 35% ASG and about 55% Portulaca extract. In another embodiment a composition in accordance with the disclosed subject matter includes about 30% ASG and about 60% Portulaca extract. In another embodiment a composition includes about 50% ASG and about 40% Portulaca extract. In another embodiment a composition includes about 55% ASG and about 35% Portulaca extract. In another embodiment a composition includes about 60% ASG and about 40% Portulaca extract.

Now referring to Table 1 an embodiment of a composition in accordance with the present disclosure is provided.

TABLE 1
Syner-C ™ Portulaca/ASG Formulation (Presperse Corp.)

| Ingredient | % w/w |
|---|---|
| Portulaca Extract | 49.5 |
| ASG | 40.00 |
| NaOH (50%) | 9.00 |
| Euxyl 9010 (Phenoxyethanol and Ethylhexylglycerin) | 1.00 |
| SHAJIFEN Sea Buckthorn Phenolic Extract | 0.5 |
| | pH - 4.5-6.0 |

The composition summarized in Table 1 provides enhanced skin whitening properties of ASG due to the synergistic effect of the combination with Portulaca extract. The composition may be added to a product formulation in amount of 0.1%-50% of the total formulation weight. The pH of the composition can be adjusted by those skilled in art to 2.5-8.5 and stabilized. The composition may be included as an additive to another formulation or on its own in a suitable carrier such as in a gel, cream or lotion for topical application.

Experiments

Experiments were conducted to evaluate suppression of melanin synthesis using B16 melanoma cells by Portulaca Extract, ASG and Portulaca/ASG blend with and without UV stress at different levels. These experiments were performed to quantify skin whitening effects of the blend versus the individual components of the blend.

Test (I)—Melanin Formation Inhibition (without UV Stress)

### Test Method

Two sets of B16-F10 melanoma cells (ATCC 6475) were seeded at 6 well multi-plates with 1×10⁵ density and cultured in Culbecco's Modified Eagle's Medium (DMEM) supplemented 10% fetal bovine serum (FBS) at 37° C. for 24 hours. The medium was exchanged with fresh DMEM media containing 10% FBS and test samples were added and cultured for 72 hours at 37° C. One set of cultured cells was washed with 2 ml of PBS (phosphate buffered saline) and cells were dissolved by 250 ul of 1N NaOH and melanin content was measured by UV absorbance at 450 nm. The other set of cells was incubated after adding 200 ul of MTT (2.5 mg/ml) at 37° C. for 4 hours. Culture medium was discarded and cells were dissolved for 10 minutes at room temperature by adding 2 ml of DMSO. After the cells were completely dissolved, UV absorbance was measured at 570 nm.

### Test Samples:

As shown in Table 2, samples used were as follows: Portulaca Extract in water, a blend of Portulaca Extract/ASG Solution, and an ASG Solution in water, in which the blend formulation was used except in place of Portulaca Extract, distilled water was added.

TABLE 2

| sample | Concentration | | | | | | | | | | | | |
|---|---|---|---|---|---|---|---|---|---|---|---|---|---|
| ASG | □ | — | 4 | 5 | — | 8 | 10 | 12 | 15 | 20 | 24 | 30 | 60 |
| | % | — | 0.2 | 0.25 | — | 0.4 | 0.5 | 0.6 | 0.75 | 1 | 1.2 | 1.5 | 3 |
| | mg/ml | — | 2.45 | 3.06 | — | 4.9 | 6.12 | 7.34 | 9.18 | 12.24 | 14.69 | 18.36 | 36.72 |
| Portulaca | □ | — | 5 | — | 8 | 10 | 12 | 15 | 20 | 24 | 30 | — | 60 |
| Extracts | % | — | 0.25 | — | 0.4 | 0.5 | 0.6 | 0.75 | 1 | 1.2 | 1.5 | — | 3 |
| | mg/ml | — | 2.54 | — | 4.06 | 5.07 | 6.08 | 7.61 | 10.14 | 12.17 | 15.21 | | 30.42 |
| Blend | □ | 8 | 10 | 12 | 15 | 20 | 24 | 30 | — | — | 60 | — | — |
| | % | 0.4 | 0.5 | 0.6 | 0.75 | 1 | 1.2 | 1.5 | — | — | 3 | — | — |
| | mg/ml | 4.94 | 6.17 | 7.4 | 9.26 | 12.34 | 14.81 | 18.51 | — | — | 37.02 | — | — |

### Test Result

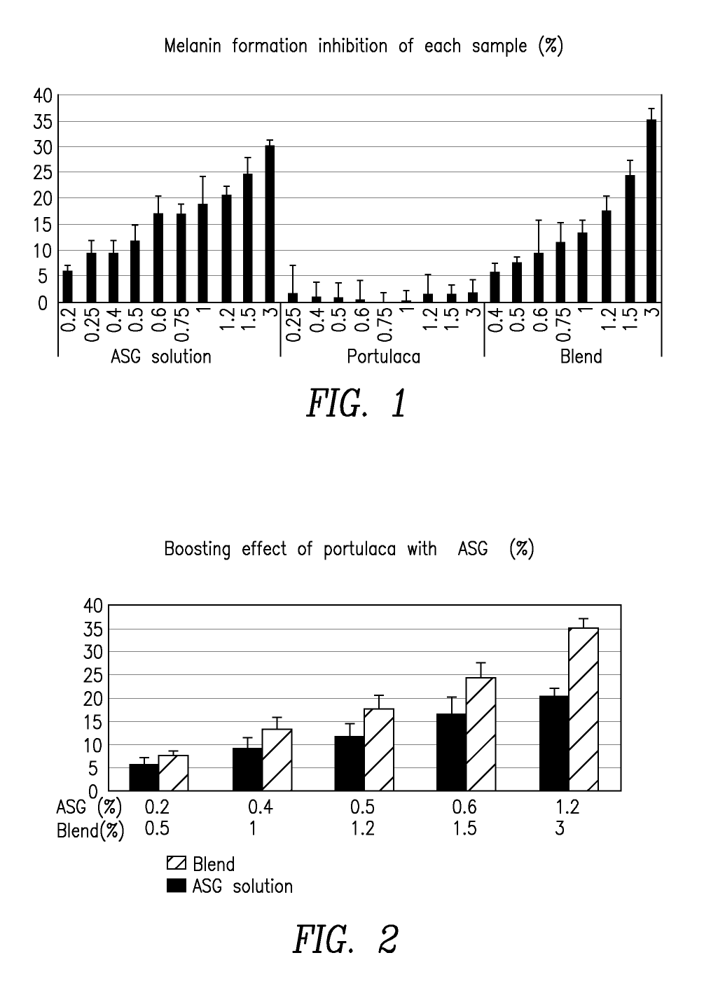

Now referring to FIGS. 1 and 2, a 3% concentration blend containing 1.2% of ASG showed about 35% of inhibition while 1.2% of ASG showed about 20% of melanin formation inhibition. Therefore, the blend with ASG and Portulaca Extract showed about a 15% increased inhibition of melanin formation. This result is unexpected due to the lack of effect shown in the Portulaca extract samples.

### SUMMARY

ASG solution (Ascorbyl Glucoside solution in water) shows melanin formation inhibition in a dose dependent manner. Portulaca extract (solution in water) shows almost no melanin formation inhibition. The blend, which is 40% ASG powder and 50% Portulaca extract, shows the same activity as ASG solution by itself, but it only contains 40% of ASG by weight while the remainder is primarily Portulaca, which has no activity against melanin.

### EXAMPLES

The following examples show how compositions of the present invention can be used in formulated cosmetic products.

### Prophetic Example 1

### Skin Lightening Emulsion with Syner C™

| | | | |
|---|---|---|---|
| Trade name | INCI name | Supplier | % w/w |

Phase A

| | | |
|---|---|---|
| Water | | QS |
| Propylene Glycol | | 2.0 |
| Ultrez 20 | Lubrizol | 0.6 |
| Na2EDTA | | 0.1 |

Phase B

| | | | |
|---|---|---|---|
| Siclone SR-5 | | Presperse | 5.0 |
| Ceraphyl 368 | Octyl Palmitate | ISP | 3.0 |
| | Myristyl Myristate | Lipo | 3.0 |
| Lipomuese 165 | Glycery Stearate and | Lipo | 3.25 |
| | PEG-100 stearate | | |
| Ceteryl Alcohol | Ceteryl alcohol | Croda | 0.75 |
| Jeecol CA-20 | Ceteth-20 | Jeen | 0.4 |

Phase C

| | |
|---|---|
| TEA (99%) | 0.6 |

Phase D

| | | |
|---|---|---|
| Euxyl PE 9010 | Schulke & Mayr | 1.0 |
| Syner-C ™ | Presperse | 5.0 |

The composition of Example 1 may be prepared as follows: disperse Ultrez 20 in water, mix all Phase A ingredients until uniform and heat the mixture to 60° C. Mix phase B ingredients and melt with mixing until uniform, maintaining the temperature between 60° C. and 70° C. Add phase B mixture to phase A while mixing, add Phase C and mix until uniform. Cool the mixture to 45° C. and add phase D ingredients to the mixture. The formulation preferably has a pH of about 6.0. The pH may be adjusted if necessary.

### Prophetic Example 2

### Skin Lightening Gel with Syner C™

| Ingredient | % w/w | Supplier |
|---|---|---|
| Water | QS | |
| Na2EDTA | 0.1 | |
| Butylene Glycol | 2.0 | |
| Syner C ™ | 5.0 | Presperse |
| Zilgel VV | 20.0 | Presperse |
| Euxyl PE 9010 | 1.0 | Schulke & Mayr |

The composition of Example 2 may be prepared by mixing all ingredients in order until uniform. The formulation preferably has a pH of about 5.5. The pH may be adjusted if necessary.

### Prophetic Example 3

### Skin Lightening Toner with Syner C™

| Ingredient | % w/w | Supplier |
|---|---|---|
| Water | QS | |
| Na2EDTA | 0.1 | |
| Butylene glycol | 2.0 | |
| Syner C ™ | 5.0 | Presperse |
| EtOH | 10.0 | |
| Euxyl PE 9010 | 1.0 | Schulke & Mayr |
| Blue #1 (1% solution) | 0.05 | |

The composition of Example 3 may be prepared by mixing all ingredients in order until uniform. The formulation preferably has a pH of about 6.5. The pH may be adjusted if necessary.

### Example 4

### Skin Lightening Emulsion with Ascorbyl PM/Portulaca Blend

| | | | |
|---|---|---|---|
| Trade name | INCI name | Supplier | % w/w |

Phase A

| | | |
|---|---|---|
| Water | | QS |
| Propylene Glycol | | 2.0 |
| Ultrez 20 | Lubrizol | 0.6 |
| Na2EDTA | | 0.1 |

Phase B

| | | | |
|---|---|---|---|
| Siclone SR-5 | | Presperse | 5.0 |
| Ceraphyl 368 | Octyl Palmitate | ISP | 3.0 |
| | Myristyl Myristate | Lipo | 3.0 |
| Lipomuese 165 | Glycery Stearate and | Lipo | 3.25 |
| | PEG-100 stearate | | |
| Ceteryl Alcohol | Ceteryl alcohol | Croda | 0.75 |
| Jeecol CA-20 | Ceteth-20 | Jeen | 0.4 |

Phase C

| | |
|---|---|
| TEA (99%) | 0.6 |

Phase D

| | | |
|---|---|---|
| Euxyl PE 9010 | Schulke & Mayr | 1.0 |
| Ascorbyl PM/ | Presperse | 5.0 |
| Portulaca blend | | |

The composition of Example 4 may be prepared as follows: disperse Ultrez 20 in water and mix until uniform. Heat the mixture to 60° C. Mix phase B ingredients and melt with mixing until uniform, maintaining the temperature between 60° C. and 70° C. Add phase B to phase C while mixing. Add Phase C and mix until uniform. Cool to 45° C. and add phase D ingredients. The formulation preferably has a pH of about 6.0. The pH may be adjusted if necessary.

### Example 5

### Skin Lightening Emulsion with Ethyl Ascorbic Acid/Portulaca Blend

| | | | |
|---|---|---|---|
| Trade name | INCI name | Supplier | % w/w |

Phase A

| | | |
|---|---|---|
| Water | | QS |
| Propylene Glycol | | 2.0 |
| Ultrez 20 | Lubrizol | 0.6 |
| Na2EDTA | | 0.1 |

Phase B

| | | | |
|---|---|---|---|
| Siclone SR-5 | | Presperse | 5.0 |
| Ceraphyl 368 | Octyl Palmitate | ISP | 3.0 |
| | Myristyl Myristate | Lipo | 3.0 |
| Lipomuese 165 | Glycery Stearate and | Lipo | 3.25 |
| | PEG-100 stearate | | |
| Ceteryl Alcohol | Ceteryl alcohol | Croda | 0.75 |
| Jeecol CA-20 | Ceteth-20 | Jeen | 0.4 |

Phase C

| | |
|---|---|
| TEA (99%) | 0.6 |

Phase D

| | | |
|---|---|---|
| Euxyl PE 9010 | Schulke & Mayr | 1.0 |
| Corum 9515/ | Presperse | 5.0 |
| Portulaca blend | | |

The composition of Example 5 may be prepared as follows: disperse Ultrez 20 in water and mix until uniform. Heat the mixture to 60° C. Mix phase B ingredients and melt with mixing until uniform, maintaining the temperature between 60° C. and 70° C. Add phase B to phase C while mixing. Add Phase C and mix until uniform. Cool to 45° C. and add phase D ingredients. The formulation preferably has a pH of about 6.0. The pH may be adjusted if necessary.

In further embodiments, compositions of the present invention may be added to various cosmetic preparations such as but not limited to sunscreen formulations, moisturizing creams/lotions, cold cream, etc.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

What is claimed is:

1. A composition comprising Portulaca extract and a vitamin C derivative selected from ascorbyl glucoside (ASG), magnesium ascorbyl phosphate and ethyl ascorbic acid.

2. The composition according to claim 1 wherein the vitamin C derivative comprises ASG.

3. The composition according to claim 1 wherein the vitamin C derivative comprises magnesium ascorbyl phosphate.

4. The composition according to claim 1 wherein the vitamin C derivative comprises and ethyl ascorbic acid.

5. The composition according to claim 1 wherein the Portulaca extract comprises about 0.2% to 1.0% by weight Portulaca Oleracea.

6. The composition according to claim 1 wherein the Portulaca extract comprises about 50% to about 99% by weight of water.

7. The composition according to claim 1 wherein the Portulaca extract further comprises one or more glycols present in an amount of about 1.0 to about 50% by weight.

8. The composition according to claim 7 wherein the one or more glycols are selected from the group consisting of butylene glycol, propylene glycol, ethylene glycol and glycerin.

9. The composition according to claim 1 comprising Portulaca extract containing about 0.2 to 0.6% by weight Portulaca Oleracea, up to 70% by weight water and up to 30% by weight butylene glycol.

10. The composition according to claim 1 further comprising at least one antioxidant.

11. The composition according to claim 10 wherein the at least one antioxidant is selected from the group consisting of Sea Buckthorn berry extract, green tea extract, ferulic acid and coenzyme Q10.

12. The composition according to claim 1 further comprising at least one preservative.

13. The composition according to claim 12 wherein the at least one preservative is selected from the group consisting of phenoxyethanol, a mixture of phenoxyethanol and ethylhexylgycerin, propylene glycol, butylene glycol, and potassium sorbate.

14. The composition according to claim 1 further comprising a base.

15. The composition according to claim 1 further comprising at least one anti-inflammatory agent.

16. The composition according to claim 15 wherein the anti-inflammatory agent comprises one or more of bisabolol, apple extract, meadowfoam extract, honeysuckle extract, and sea whip extract.

17. The composition according to claim 1 comprising from about 2-98% by weight vitamin C derivative and from about 2-98% by weight Portulaca extract.

18. The composition according to claim 1 comprising from about 20%-60% vitamin C derivative and about 40%-80% Portulaca extract.

19. The composition according to claim 1 comprising about 45% vitamin C derivative and about 45% Portulaca extract.

20. The composition according to claim 1 comprising about 40% vitamin C derivative and about 49 to about 50% Portulaca extract.

21. The composition according to claim 1 comprising about 35% vitamin C derivative and about 55% Portulaca extract.

22. The composition according to claim 1 comprising about 30% vitamin C derivative and about 60% Portulaca extract.

23. The composition according to claim 1 comprising about 50% vitamin C derivative and about 40% Portulaca extract.

24. The composition according to claim 1 comprising about 55% vitamin C derivative and about 35% Portulaca extract.

25. The composition according to claim 1 comprising about 60% vitamin C derivative and about 30% Portulaca extract.

26. The composition according to claim 20 wherein the vitamin C derivative comprises ASG.

27. The composition according to claim 26 further comprising about 9.0% by weight NaOH.

28. An additive for a cosmetic or pharmaceutical formulation, the additive comprising 49.5% by weight Portulaca extract containing 0.2 to 0.6% by weight Portulaca Oleracea and 40% by weight ASG, the additive having a pH of about 4.5-6.
